(19) United States
(12) Patent Application Publication
(10) Pub. No.: US 2007/0042406 A1
(43) Pub. Date: Feb. 22, 2007

(54) Diffusion mediated clean-up of a target carrier fluid

(75) Inventors: Gregory R. Yantz, Cambridge, MA (US); Jonathan W. Larson, New Ipswich, NH (US); Rudolf Gilmanshin, Waltham, MA (US)

Correspondence Address: WOLF GREENFIELD & SACKS, PC, FEDERAL RESERVE PLAZA, 600 ATLANTIC AVENUE, BOSTON, MA, 02210-2206, US

(73) Assignee: U.S. Genomics, Inc., Woburn, MA (US)

(21) Appl. No.: 11/486,446

(22) Filed: Jul. 13, 2006

Related U.S. Application Data
(60) Provisional application No. 60/700,689, filed on Jul. 18, 2005.

(51) Int. Cl. C12Q 1/68; C12M 3/00

(52) U.S. Cl. 435/6; 435/287.2

(57) ABSTRACT

Microfludic channels are constructed for use in preparing and/or analyzing samples. In one embodiment, a microfluidic channel receives a carrier fluid having both non-targets and targets. The non-targets are moved from the carrier fluid by diffusion and into sheathing fluids also present in the channel before contents of the carrier fluid are analyzed.

### RELATED APPLICATIONS

This application claims priority under 35 U.S.C. § 119(e) to U.S. Provisional Application Ser. No. 60/700,689, filed on Jul. 18, 2005, which is hereby incorporated by reference in its entirety.

### BACKGROUND OF INVENTION

1. Field of Invention

The invention relates to manipulating a sample, such as a sample that includes biological polymers, and more particularly to manipulating the sample in a microfluidic channel for subsequent analysis.

2. Discussion of Related Art

It is now possible to detect and analyze a polymer when the polymer is in an aligned or elongated state. U.S. Pat. No. 6,355,420, which is hereby incorporated by reference in its entirety, describes methods for linear analysis of polymers. The methods described therein provide methods for rapid detection of different components that comprise the polymer.

Sequence analysis of polymers has many practical applications. Of great interest is the ability to sequence the genomes of various organisms, including the human genome. Specific sequences can be recognized with a host of sequence-specific probes such as oligonucleotides, engineered proteins, and also synthetic compounds. In these sequence-specific approaches, there is sometimes a need to resolve the position of probes relative to one another, or to other features of the polymer, in order to generate a map of the polymer.

Linear analysis of polymers, such as DNA, may be accomplished by moving a detection zone over a fixed polymer, or by moving a polymer through a detection zone. These approaches make use of instrumentation and a detection signal to acquire information from the sequence-specific probes on the polymer when they are within the detection zone. For instance, fluorescence, atomic force microscopy (AFM), scanning tunneling microscopy (STM), as well as other electrical and electromagnetic methods, are suitable for capturing signals and thereby “reading” the sequence information of a polymer.

It can be desirable to remove non-targets, such as excess and/or unbound sequence-specific probes, from a sample fluid prior to analysis of targets, such as polymers, that also reside in the sample fluid. Unbound probes may confuse and complicate the analysis. Present methods, such as dialysis, can prove time consuming. To this end, there is a need for improved methods and devices for removing non-targets from sample fluid prior to analysis of targets.

### SUMMARY OF INVENTION

According to one aspect of the invention, a microfluidic apparatus is disclosed. The microfluidic apparatus comprises a microchannel having an upstream portion and a downstream portion. The microchannel is constructed and arranged to transport a carrier fluid such that, when present in the carrier fluid, targets and non-targets flow from the upstream portion toward the downstream portion. The apparatus also comprises a first sheathing fluid introduction channel that is adapted to provide a first sheathing fluid to the microchannel such that non-targets can diffuse from the carrier fluid to the first sheathing fluid. The microfluidic apparatus also comprises a sample capture channel located downstream from the first sheathing fluid introduction channel that receives the carrier fluid after at least a portion of the non-targets have diffused from the carrier fluid and into the first sheathing fluid.

According to another aspect of the invention, a method is disclosed for removing non-targets from a carrier fluid that contains targets with the microfluidic apparatus.

In one embodiment, the sample capture channel is positioned with respect the microchannel such that at least 60% (0.60) or at least 85% (0.85) of the non-targets introduced to the microchannel in the carrier fluid are removed from the carrier fluid that passes through the sample capture channel.

In some embodiments, the sample capture channel is positioned with respect the microchannel and conditions are such that at least 80% (0.80) of the targets introduced to the microchannel in the carrier fluid are retained within the carrier fluid that passes through the sample capture channel. In another embodiment, at least 90% (0.90) of the targets introduced to the microchannel in the carrier fluid are retained within the carrier fluid.

In one emobdiment, the first sheathing fluid introduction channel comprises a pair of opposed fluid introduction channels adapted to introduce a pair of opposed flows of sheathing fluid into the microchannel. In some embodiments, the pair of opposed flows of sheathing fluid create a velocity gradient within the carrier fluid.

In some embodiments, a detection zone is located in the sample capture channel.

In some embodiments, a first fluid removal channel is adapted to remove fluid from the microchannel that is excluded from passing through the sample capture channel. In some embodiments, the sample capture channel defines portions of the first fluid removal channel. In some of such embodiments, the sample capture channel includes opposed walls of the microchannel that are downstream from the first fluid removal channel. Still, in other of such embodiments, the first fluid removal channel comprises a pair of opposed fluid removal channels. The first fluid removal channel may remove all of the first sheathing fluid from the microchannel and/or may remove a portion of the carrier fluid from the microchannel.

Some embodiments further comprise a second sheathing fluid introduction channel that provides a second sheathing fluid to the microchannel such that non-targets can diffuse from the carrier fluid to the second sheathing fluid. A detection zone may be located in the microchannel downstream from the second sheathing fluid introduction channel. The detection zone may be sized and spaced from the second sheathing fluid introduction channel such that fewer than 10% (0.10) or fewer than 5% (0.05) of the non-targets introduced to the microchannel in the carrier fluid pass through the detection zone in the microchannel.

Some of such embodiments further comprise a second fluid removal channel to remove at least a portion of the second sheathing fluid from the microchannel. The second fluid removal channel communicates with the microchannel at a position downstream from the second sheathing fluid introduction channel. The second fluid removal channel may be sized and positioned with respect to the microchannel and conditions may be such that fewer than 10% (0.10) or fewer than 5% (0.05) of the non-targets introduced to the microchannel in the carrier fluid remain in the microchannel at points downstream from the second fluid removal channel.

Some of such embodiments may further comprise a third sheathing fluid introduction channel to provide a third sheathing fluid to the microchannel at a position downstream from the second fluid removal channel such that non-targets can diffuse from the carrier fluid to the third sheathing fluid.

In some embodiments, the non-targets include unincorporated labels. The unincorporated labels may include fluorescent labels or quantum dots. The non-targets may include excess reactants or smaller reactants. The non-targets may include unbound probes. The probes may include non-hybridized oligonucleotides, enzymes, dendrimers, antibodies, aptamers or immunoglobulins.

In some embodiments, the targets include polymers. The polymers may include peptides. The peptides may be proteins. The polymers may be nucleic acids, such as DNA or RNA. The RNA may be miRNA, siRNA, or RNAi.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a plan view of a microfluidic channel that may be used in diffusion mediated cleanup of a target carrier fluid, according to one embodiment of the invention;

FIG. 2 is a graphical representation of target concentration profile and non-target concentration profile taken across line 2-2 in the embodiment of FIG. 1;

FIG. 3 is a plan view of a microchannel having a sample capture channel for use in diffusion mediated cleanup of a target carrier fluid, according to one embodiment;

FIG. 4 is a plan view of another embodiment of a sample capture channel; and

FIG. 5 is a plan view of an embodiment of a microfluidic channel used during an experiment relating to diffusion mediated cleanup of a carrier fluid.

### DETAILED DESCRIPTION

According to a first aspect of the invention, a microchannel can be adapted such that non-targets are excluded from a carrier fluid flowing there through prior to analysis of targets also within the carrier fluid. The microchannel receives a carrier fluid including both targets to be analyzed and non-targets that are preferably excluded from the carrier fluid prior to analysis of the targets. A sheathing fluid that lacks non-targets is provided to the microchannel. As the carrier fluid and sheathing fluid move through the microchannel, non-targets diffuse from the carrier fluid to the sheathing fluid more rapidly than the targets. Thus, as the carrier fluid moves downstream, the concentration of non-targets decreases more rapidly than the concentration of targets. A sample capture channel is located downstream in the microchannel to capture the carrier fluid after the concentration of non-targets has decreased greater than the concentration of targets.

As used herein the terms “microchannel” and/or “microfluidic channel” refer to a channel having an average cross sectional area, taken in the direction perpendicular to flow, that is fewer than 25 square millimeters. It is to be appreciated that some portions of the channel can have cross sectional areas larger than 25 square millimeters. It is also to be appreciated that many embodiments can have microchannels with average cross sectional areas that are much smaller than 25 square millimeters. By way of example, some embodiments may have portions with cross sectional areas that are less than 1 square millimeter, less than 500 microns, less than 100 microns, and smaller, as the term microchannel implies no lower bound on the size that the channel can have.

As used herein, the term “targets” refers to entities within a carrier fluid passing through the microchannel that are to be analyzed. In some embodiments the targets are polymers, such as DNA or RNA that are provided in the carrier fluid to the microchannel. The polymers are directed to a device downstream from the microchannel, or a detection zone within the microchannel to be analyzed. It is to be appreciated that the term “targets” may refer to other types of entities that are to be analyzed, such as molecules, cells, and the like, as targets are not limited to polymers.

As the term is used herein, “non-targets” refers to entities within a carrier fluid that are preferably excluded from the carrier fluid prior to analysis that is performed on the targets. By way of example, in some embodiments where the targets are polymers, probes are introduced to the carrier fluid such that some of the probes associate themselves with the polymers in specific manners. The probes, once associated with the polymers, are then detected such that the position of the probes relative to the polymers or other probes also located on the polymer can provide information about the polymer. Probes that do associate themselves with the polymer non-specifically as well as probes in close proximity to the polymer can also be detected and may confuse the analysis of the polymer. To this end, it is preferable to exclude probes that are not associated with polymers prior to analysis. It is to be appreciated that although non-targets may comprise probes, other entities, such as nucleotides, enzymes, quantum dots, and the like may also comprise non-targets as aspects of the invention are not limited in this regard.

Both targets and non-targets diffuse about fluids, such as a carrier fluid, in a stochastic manner according to the laws of diffusion. Eventually, this results in a uniform concentration of the targets and non-targets throughout the fluid, although the targets and non-targets may still be moving about the fluid even after an equilibrium concentration is reached. The rate at which the targets and non-targets diffuse throughout the fluid is controlled by numerous factors, including the size of the elements, the shape of the elements, and other factors normally associated with diffusion of particles within a fluid. Targets, which are typically larger than the non-targets, generally diffuse more slowly from a carrier fluid to a sheathing fluid than non-targets.

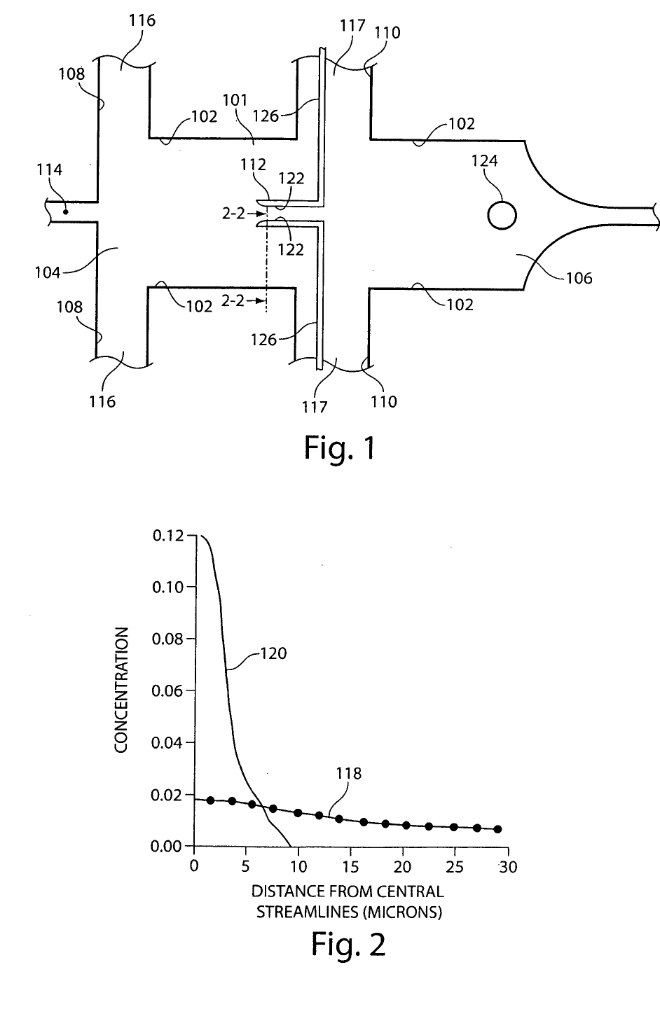

FIG. 1 is a schematic of a microchannel 101 having opposed walls 102, an upstream portion 104, a downstream portion 106, a pair of sheathing fluid introduction channels 108 and a sample capture channel 112. The microchannel receives a carrier fluid 114 containing both targets and non-targets near the upstream portion of the microchannel. “Carrier fluid” as the term is used herein, refers to any fluid that includes targets when provided to the microchannel. Sheathing fluids 116 or side flows, which lack or at least have lower concentrations of non-targets, are introduced near the upstream portion 104 of the microchannel and flow towards the downstream portion 106 alongside the carrier fluid 114. The sample capture channel 112, located downstream in the microchannel, captures at least a portion of the carrier fluid 114 after the carrier fluid has traveled in the microchannel alongside the sheathing fluids 116 such that the concentration of non-targets in the carrier fluid is reduced more than the concentration of targets. As used herein, the term “sheathing fluid” refers to any fluid introduced to the microchannel other than the carrier fluid.

As the carrier and sheathing fluids progress towards the downstream portion of the microchannel, the targets and non-targets in the carrier fluid diffuse laterally toward the adjacent sheathing fluids in the microchannel. As shown in FIG. 2, the non-targets diffuse more rapidly than the targets, such that the concentration of non-targets 118 in the carrier fluid decreases greater than the concentration of targets 120 in the carrier fluid. FIG. 2 shows a concentration profile for both targets and non-targets, taken laterally across half of the microchannel along lines 2-2 of FIG. 1. While FIG. 2 represents one lateral side of the microchannel, diffusion across the opposite side of the microchannel should follow a similar pattern due to the symmetrical nature of the microchannel and fluids passing therethrough. As can be seen, the non-targets have diffused from the carrier fluid more rapidly than the targets. In fact, very few of the targets have diffused more than one third of the way into either sheathing fluid while the non-targets have diffused to a near homogenous concentration across the microchannel.

As mentioned above, the carrier fluid or at least a portion thereof is passed through a sample capture channel 112 at some point after the concentration of non-targets 118 has decreased greater than the concentration of targets 120. In the embodiment shown in FIG. 1, the sample capture channel 112 includes a pair of opposed walls 122 within a downstream portion of the microchannel. The sample capture channel of FIG. 1 physically segregates a portion of the fluid passing through the microchannel that has a concentration of non-targets reduced more than a concentration of targets. In this regard, the sample capture channel prevents further mixing between fluid passing therethrough and fluid passing around the sample capture channel. The opposed walls 122 of the sample capture channel shown in FIG. 1 are funnel shaped, and create a velocity gradient in fluid passing there through, so as to focus the contents of the carrier fluid. However, it is to be appreciated that sample capture channels may comprise different types of structures, as is discussed in greater detail herein.

Concentration profiles of both targets 120 and non-targets 118 in a microchannel 101, such as that shown in FIG. 2, can be used to determine an appropriate size and placement of a sample capture channel 112 or a detection zone 124 within the microchannel. The concentration profile can be used to determine how far downstream a sample capture channel or detection zone 124 should be placed. The concentration profile can also be used to determine an appropriate width for a sample capture channel or detection zone, so as to determine how much of the carrier fluid/sheathing fluid passes through the capture channel 112 or detection zone 124. For instance, in some embodiments it may be desirable to exclude only ten percent of all of the fluid passing through the microchannel from passing through the capture channel or detection zone. In other embodiments, upwards of sixty percent of all of the fluid passing through the microchannel may be excluded from passing through the capture channel or detection zone, as aspects of the present invention are not limited in this regard. In the embodiment of FIGS. 1 and 2, the concentration profile is used to determine a width of the sample capture channel such that a high concentration of targets pass through the capture channel.

Some illustrative embodiments include fluid removal channels 126, like those depicted in FIG. 1. As shown, the fluid removal channels 126 are located adjacent the sample capture channel 112, and act to remove fluid from the microchannel 101 that does not pass through the sample capture channel. The sheathing fluid removal channels do not necessarily remove all of the sheathing fluids 116 that are provided into the microchannel, as in some embodiments portions of the sheathing fluid pass through the capture channel. Also, in some embodiments the fluid removal channels remove portions of the carrier fluid 114 that does not pass through the sample capture channel. Still, in some embodiments mixing occurs between the carrier fluid and the sheathing fluid such that some portions of the carrier fluid are removed by the removal channels, and some portions of the sheathing fluid pass through the capture channel.

Some illustrative embodiments of channels can also be used to initiate, perform and/or to control reactions. By way of example, diffusion between the carrier fluid and the sheathing fluids can be used to introduce reactants to one another in a controlled manner. A sample capture channel can be positioned appropriately such that after a certain amount of diffusion between the sheathing fluid 116 and carrier fluid 114 has occurred, further diffusion is prevented by physical separation of the fluid that passes through the sample capture channel and the remaining fluid.

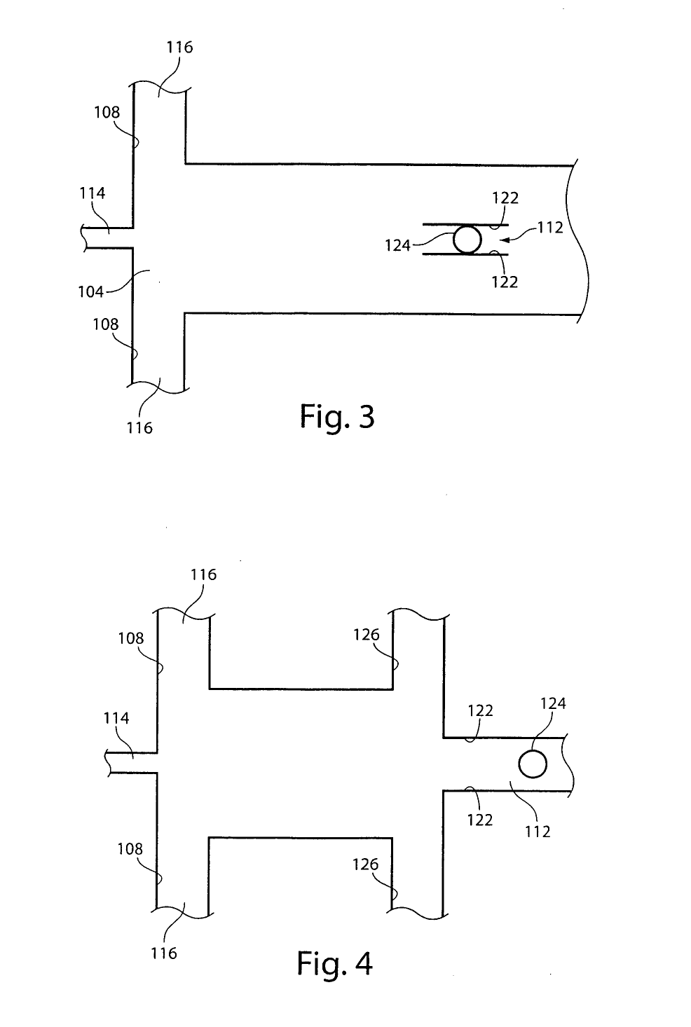

The configuration of sample capture channels and fluid removal channels are not limited to those of the embodiment shown in FIG. 1. By way of example, FIG. 3 shows an embodiment with a sample capture channel that comprises a pair of opposed walls 122 within the microchannel. Here, the fluid that passes around rather than through the capture channel is not removed from the microchannel, but rather is reintroduced to the microchannel at a point downstream of the capture channel. In the embodiment of FIG. 4, the sample capture channel comprises opposed walls 122 of the microchannel itself located at a position downstream from a pair of opposed fluid removal channels 126. Still, other embodiments have different configurations of capture channels and/or fluid removal channels, as aspects of the invention are not limited to the illustrated embodiments.

Flow characteristics of either the carrier or sheathing fluids can be altered to change the concentration profile near the sample capture channel. By way of example, in some embodiments, the flow rates of both the carrier fluid and sheathing fluid can be increased, such that the fluids will reach the capture channel in less time, thus allowing less time for diffusion to occur. In other embodiments, the sheathing fluids may be used to create a velocity gradient and elongational flow within the carrier fluid to help focus a portion of the carrier fluid into the sample capture channel. As used herein, the terms “elongational flow” and “velocity gradient” refer to flow that is accelerating as it moves downstream. Still, in some embodiments the velocity of the sheathing fluids may be altered relative to one another such that the carrier fluid can be positioned laterally within the microchannel to direct the carrier fluid into the capture channel or elsewhere. It is to be appreciated that the concentration profiles, or the effects of changing variables like fluid velocity or microchannel geometry, may be determined either experimentally or through simulation, as the invention is not limited in this regard.

A second sheathing fluid 117 or a pair of sheathing fluids can be introduced to the microchannel downstream from the fluid removal channels 26. In the embodiment of FIG. 1, a second pair of sheathing fluids 117 are introduced immediately downstream of the capture channel through a second pair of sheathing fluid introduction channels 110. Here, diffusion of non-targets from the carrier fluid to the second sheathing fluids occurs as the carrier fluid moves through the microchannel alongside the second sheathing fluids. Other embodiments can incorporate additional fluid removal channels and/or additional sheathing fluid introduction channels.

Introducing additional sheathing fluids may prove particularly beneficial in reducing the concentration of non-targets below the equilibrium that can be achieved with only the first sheathing fluid(s). For instance, FIG. 2 depicts non-targets that have nearly reached an equilibrium across the microchannel. At this point, further diffusion of non-targets to the sheathing fluid will be countered with reverse diffusion back from the sheathing fluid. However, after lateral portions of the fluid are removed by fluid removal channels, the second sheathing fluid introduction channels 110 provide sheathing fluid 117 with a much lower concentration of non-targets, or no non-targets at all. The diffusion of non-targets from the carrier fluid to the sheathing fluid will then again be greater than the diffusion of targets from the carrier fluid. In this regard, introducing additional sheathing fluids can allow reduction in the concentration of non-targets that may not be achieved without removing fluid from the microchannel.

Various embodiments of the invention can incorporate any number of sheathing fluid introduction and removal channels. In some embodiments, additional fluid can be removed from the microchannel by second fluid removal channels. Still, in some embodiments, a second sample capture channel can be incorporated into the microchannel, like that shown in FIG. 2. Still, a third or even fourth sheathing fluid introduction channels and corresponding fluid removal channels can be incorporated into some embodiments, as there is no limit to the number of introduction and removal channels that an embodiment can have.

Sequentially introducing and removing sheathing fluids to the microchannel can exponentially increase the ability of the microchannel to remove non-targets from the carrier fluid. By way of example, in one embodiment a first pair of fluid removal channels remove 75% (0.75) of the non-targets, while only removing 25% (0.25) of the targets then present. After a second pair of sheathing fluids are introduced into the microchannel, a second pair of removal channels again remove 75% (0.75) of the non-targets that are then present in the microchannel, while again only removing 25% (0.25) of the targets then present. In such an embodiment, the concentration of targets left after the second removal channel is 85% times 85%, or 72.25%, (0.85×0.85=0.7225) when measured as a percentage of the targets initially provided to the microchannel. At the same point within the microchannel, the concentration of non-targets is 25% times 25%, or 6.25% (0.25×0.25=0.0625) when measured as a percentage of the non-targets initially provided to the microchannel. A third pair of sheathing fluid introduction and removal channels having the same target and non-target removal characteristics leaves the carrier fluid with 61.41% (0.6141) of the targets initially provided to the microchannel and only 1.56% (0.0156) of the non-target initially provided to the microchannel.

Detection zones 124 can be placed at various positions within the microchannel 101. In the embodiment of FIG. 1, detection zones 124 are located both near a central portion of the microchannel 101 at a point downstream from the second pair of sheathing fluid introduction channels 110 and within the first sample capture channel 112. It is to be appreciated that such detection zones can be placed across only a portion of the microchannel or capture channel, or across the entire microchannel or capture channel. Concentration profiles like those of FIG. 2 can be used to help determine optimal placement and sizes of such detection zones. In the embodiment of FIG. 3, a detection zone is disposed across the sample capture channel, such that the entire contents of the fluid passing therethrough also pass through the detection zone. In the embodiment of FIG. 4, a detection zone 124 is disposed across a portion of the sample capture channel 112. Here, prior to passing through the detection zone, additional non-targets diffuse from the carrier fluid into the sheathing fluid at a greater rate than the targets. In this manner, a portion of the non-targets diffuse away from central portions of the microchannel and do not pass through the detection zone while most of the targets remain in a central portion and do pass through the detection zone.

In some embodiments, sheathing fluids may include non-targets that are to diffuse into the carrier fluid. As described above, non-targets may be exposed to targets in the carrier fluids such that they can associate with the target, if appropriate for subsequent analysis of the target. In this regard, sequential sheathing fluid introduction and removal channels can also be used to introduce and subsequently remove non-targets for this purpose. As used herein, the term “plurality” when used with reference to targets or non-targets refers to up to an infinite number of targets or non-targets. However, in some embodiments, plurality denotes fewer than 10⁸, fewer than 10⁶, fewer than 10⁴, and even as few as 2.

Microfluidic devices associated with diffusion mediated cleanup can be used with other microfluidic devices, such as any of those described in U.S. patent application Ser. No. 10/821,664 titled Advanced Microfluidics, now published as US 2005-0112606 A1.

Samples can be derived from virtually any source known or suspected to contain an agent of interest. Samples can be of solid, liquid or gaseous nature. They may be purified but usually are not. Different samples can be collected from different environments and prepared in the same manner by using the appropriate collecting device.

The samples to be tested can be a biological or bodily sample such as a tissue biopsy, urine, sputum, semen, stool, saliva and the like. The invention further contemplates preparation and analysis of samples that may be biowarfare targets. Air, liquids and solids that will come into contact with the greatest number of people are most likely to be biowarfare targets. Samples to be tested for the presence of such agents may be taken from an indoor or outdoor environment. Such biowarfare sampling can occur continuously, although this may not be necessary in every application. For example, in an airport setting, it may only be necessary to harvest randomly a sample near or around select baggage. In other instances, it may be necessary to continually monitor (and thus sample the environment). These instances may occur in “heightened alert” states. In some important embodiments, the sample is tested for the presence of a pathogen. Examples include samples to be tested for the presence of a pathogenic substances such as but not limited to food pathogens, water-borne pathogens, and aerosolized pathogens.

Liquid samples can be taken from public water supplies, water reservoirs, lakes, rivers, wells, springs, and commercially available beverages. Solids such as food (including baby food and formula), money (including paper and coin currencies), public transportation tokens, books, and the like can also be sampled via swipe, wipe or swab testing and placing the swipe, wipe or swab in a liquid for dissolution of any agents attached thereto. Based on the size of the swipe or swab and the volume of the corresponding liquid it must be placed in for agent dissolution, it may or may not be necessary to concentrate such liquid sample prior to further manipulation.

Air samples can be tested for the presence of normally airborne substances as well as aerosolized (or weaponized) chemicals or biologics that are not normally airborne. Air samples can be taken from a variety of places suspected of being biowarfare targets including public places such as airports, hotels, office buildings, government facilities, and public transportation vehicles such as buses, trains, airplanes, and the like.

Analysis of samples may embrace the use of one or more reagents (i.e., at least one reagent) that acts on or reacts with and thereby modifies a target agent. At least one reagent however is less than an infinite number of reagents as used herein and more commonly represents less than 1000, less than 100, less than 50, less than 20, less than 10 or less than 5. The nature of the reagents will vary depending on the analysis being performed using such reagent. The reagent may be a lysing agent (e.g., a detergent such as but not limited to deoxycholate), a labeling agent or probe (e.g., a sequence-specific nucleic acid probe), an enzyme (e.g., a nuclease such as a restriction endonuclease), an enzyme co-factor, a stabilizer (e.g., an anti-oxidant), and the like. One of ordinary skill in the art can envision other reagents to be used in the invention.

Additionally, the fluids used in the invention may contain other components such as buffering compounds (e.g., TRIS), chelating compounds (e.g., EDTA), ions (e.g., monovalent, divalent or trivalent cations or anions), salts, and the like.

The invention is not limited in the nature of the agent being analyzed (i.e., the target agent). These agents include but are not limited to cells and cell components (e.g., proteins and nucleic acids), chemicals and the like. These agents may be biohazardous agents. Target agents may be naturally occurring or non-naturally occurring, and this includes agents synthesized ex vivo but released into a natural environment. A plurality of agents is more than one and less than an infinite number. It includes less than 10¹⁰, less than 10⁹, less than 10⁸, less than 10⁹, less than 10⁷, less than 10⁶, less than 10⁵, less than 10⁴, less than 5000, less than 1000, less than 500, less 100, less than 50, less than 25, less than 10 and less than 5, as well as every integer therebetween as if explicitly recited herein.

A “polymer” as used herein is a compound having a linear backbone to which monomers are linked together by linkages. The polymer is made up of a plurality of individual monomers. An individual monomer as used herein is the smallest building block that can be linked directly or indirectly to other building blocks (or monomers) to form a polymer. At a minimum, the polymer contains at least two linked monomers. The particular type of monomer will depend upon the type of polymer being analyzed. The polymer may be a nucleic acid, a peptide, a protein, a carbohydrate, an oligo- or polysaccharide, a lipid, etc. The polymer may be naturally occurring but it is not so limited.

In some embodiments, the polymer is capable of being bound to or by sequence- or structure-specific probes, wherein the sequence or structure recognized and bound by the probe is unique to that polymer or to a region of the polymer. It is possible to use a given probe for two or more polymers if a polymer is recognized by two or more probes, provided that the combination of probes is still specific for only a given polymer. A sample containing polymers, in some instances, can be analyzed as is without harvest and isolation of polymers contained therein.

In some embodiments, the method can be used to detect a plurality of different polymers in a sample.

As used herein, stretching of the polymer means that the polymer is provided in a substantially linear extended form rather than a compacted, coiled and/or folded form.

In some important embodiments, the polymers are nucleic acids. The term “nucleic acid” refers to multiple linked nucleotides (i.e., molecules comprising a sugar (e.g., ribose or deoxyribose) linked to an exchangeable organic base, which is either a pyrimidine (e.g., cytosine (C), thymidine (T) or uracil (U)) or a purine (e.g., adenine (A) or guanine (G)). “Nucleic acid” and “nucleic acid molecule” are used interchangeably and refer to oligoribonucleotides as well as oligodeoxyribonucleotides. The terms shall also include polynucleosides (i.e., a polynucleotide minus a phosphate) and any other organic base containing nucleic acid. The organic bases include adenine, uracil, guanine, thymine, cytosine and inosine.

In important embodiments, the nucleic acid is DNA or RNA. DNA includes genomic DNA (such as nuclear DNA and mitochondrial DNA), as well as in some instances complementary DNA (cDNA). RNA includes messenger RNA (mRNA), miRNA, and the like. The nucleic acid may be naturally or non-naturally occurring. Non-naturally occurring nucleic acids include but are not limited to bacterial artificial chromosomes (BACs) and yeast artificial chromosomes (YACs). Harvest and isolation of nucleic acids are routinely performed in the art and suitable methods can be found in standard molecular biology textbooks. (See, for example, Maniatis' Handbook of Molecular Biology.) Preferably, prior amplification using techniques such as polymerase chain reaction (PCR) are not necessary. Accordingly, the polymer may be a non in vitro amplified nucleic acid. As used herein, a “non in vitro amplified nucleic acid” refers to a nucleic acid that has not been amplified in vitro using techniques such as polymerase chain reaction or recombinant DNA methods. A non in vitro amplified nucleic acid may however be a nucleic acid that is amplified in vivo (in the biological sample from which it was harvested) as a natural consequence of the development of the cells in vivo. This means that the non in vitro nucleic acid may be one which is amplified in vivo as part of locus amplification, which is commonly observed in some cell types as a result of mutation or cancer development.

As used herein with respect to linked units of a polymer including a nucleic acid, “linked” or “linkage” means two entities bound to one another by any physicochemical means. Any linkage known to those of ordinary skill in the art, covalent or non-covalent, is embraced. Natural linkages, which are those ordinarily found in nature connecting for example the individual units of a particular nucleic acid, are most common. Natural linkages include, for instance, amide, ester and thioester linkages. The individual units of a nucleic acid analyzed by the methods of the invention may be linked, however, by synthetic or modified linkages. Nucleic acids where the units are linked by covalent bonds will be most common but those that include hydrogen bonded units are also embraced by the invention. It is to be understood that all possibilities regarding nucleic acids apply equally to nucleic acid targets and nucleic acid probes, as discussed herein.

The nucleic acids may be double-stranded, although in some embodiments the nucleic acid targets are denatured and presented in a single-stranded form. This can be accomplished by modulating the environment of a double-stranded nucleic acid including singly or in combination increasing temperature, decreasing salt concentration, and the like. Methods of denaturing nucleic acids are known in the art.

Target nucleic acids (i.e., those of interest) commonly have a phosphodiester backbone because this backbone is most common in vivo. However, they are not so limited. Backbone modifications are known in the art. One of ordinary skill in the art is capable of preparing such nucleic acids without undue experimentation. The probes, if nucleic acid in nature, can also have backbone modifications such as those described herein.

Thus the nucleic acids may be heterogeneous in backbone composition thereby containing any possible combination of nucleic acid units linked together such as peptide nucleic acids (which have amino acid linkages with nucleic acid bases, and which are discussed in greater detail herein). In some embodiments, the nucleic acids are homogeneous in backbone composition.

Probes may be used to analyze polymers. As used herein, a probe is a molecule or compound that binds preferentially to the agent (e.g., a polymer) of interest (i.e., it has a greater affinity for the agent of interest than for other compounds). Its affinity for the agent of interest may be at least 2-fold, at least 5-fold, at least 10-fold, or more than its affinity for another compound. Probes with the greatest differential affinity are preferred in most embodiments. Binding of a probe to an agent may indicate the presence and location of a target site in the target agent, or it may simply indicate the presence of the agent, depending on user requirements. As used herein, a target agent that is bound by a probe is “labeled” with the probe and/or its detectable label.

The probes can be of any nature including but not limited to nucleic acid (e.g., aptamers), peptide, carbohydrate, lipid, and the like, or some combination thereof. A nucleic acid based probe such as an oligonucleotide can be used to recognize and bind DNA or RNA. The nucleic acid based probe can be DNA, RNA, LNA or PNA, although it is not so limited. It can also be a combination of one or more of these elements and/or can comprise other nucleic acid mimics. With the advent of aptamer technology, it is possible to use nucleic acid based probes in order to recognize and bind a variety of compounds, including peptides and carbohydrates, in a structurally, and thus sequence, specific manner. Other probes for nucleic acid targets include but are not limited to sequence-specific major and minor groove binders and intercalators, nucleic acid binding peptides or proteins, etc.

As used herein a “peptide” is a polymer of amino acids connected preferably but not solely with peptide bonds. The probe may be an antibody or an antibody fragment. Antibodies include IgG, IgA, IgM, IgE, IgD as well as antibody variants such as single chain antibodies. Antibody fragments contain an antigen-binding site and thus include but are not limited to Fab and F(ab)₂ fragments.

The probes may bind to the target polymer in a sequence-specific manner. “Sequence-specific” when used in the context of a nucleic acid means that the probe recognizes a particular linear (or in some instances quasi-linear) arrangement of nucleotides or derivatives thereof. In some embodiments, the probes are “polymer-specific” meaning that they bind specifically to a particular polymer, possibly by virtue of a particular sequence or structure unique to that polymer.

In some instances, nucleic acid probes will form at least a Watson-Crick bond with a target nucleic acid. In other instances, the nucleic acid probe can form a Hoogsteen bond with the target nucleic acid, thereby forming a triplex. A nucleic acid probe that binds by Hoogsteen binding enters the major groove of a nucleic acid polymer and hybridizes with the bases located there. Examples of these latter probes include molecules that recognize and bind to the minor and major grooves of nucleic acids (e.g., some forms of antibiotics). In some embodiments, the nucleic acid probes can form both Watson-Crick and Hoogsteen bonds with the nucleic acid polymer. BisPNA probes, for instance, are capable of both Watson-Crick and Hoogsteen binding to a nucleic acid.

The nucleic acid probes of the invention can be any length ranging from at least 4 nucleotides to in excess of 1000 nucleotides. In preferred embodiments, the probes are 5-100 nucleotides in length, more preferably between 5-25 nucleotides in length, and even more preferably 5-12 nucleotides in length. The length of the probe can be any length of nucleotides between and including the ranges listed herein, as if each and every length was explicitly recited herein. Thus, the length may be at least 5 nucleotides, at least 10 nucleotides, at least 15 nucleotides, at least 20 nucleotides, or at least 25 nucleotides, or more, in length. The length may range from at least 4, at least 5, at least 6, at least 7, at least 8, at least 9, at least 10, at least 12, at least 15, at least 20, at least 25, at least 50, at least 75, at least 100, at least 150, at least 200, at least 250, at least 500, or more nucleotides (including every integer therebetween as if explicitly recited herein).

It should be understood that not all residues of the probe need hybridize to complementary residues in the nucleic acid target. For example, the probe may be 50 residues in length, yet only 25 of those residues hybridize to the nucleic acid target. Preferably, the residues that hybridize are contiguous with each other.

The probes are preferably single-stranded, but they are not so limited. For example, when the probe is a bisPNA it can adopt a secondary structure with the nucleic acid polymer resulting in a triple helix conformation, with one region of the bisPNA clamp forming Hoogsteen bonds with the backbone of the polymer and another region of the bisPNA clamp forming Watson-Crick bonds with the nucleotide bases of the polymer.

The nucleic acid probe hybridizes to a complementary sequence within the nucleic acid polymer. The specificity of binding can be manipulated based on the hybridization conditions. For example, salt concentration and temperature can be modulated in order to vary the range of sequences recognized by the nucleic acid probes. Those of ordinary skill in the art will be able to determine optimum conditions for a desired specificity.

In some embodiments, the probes may be molecular beacons. When not bound to their targets, the molecular beacon probes form a hairpin structure and do not emit fluorescence since one end of the molecular beacon is a quencher molecule. However, when bound to their targets, the fluorescent and quenching ends of the probe are sufficiently separated so that the fluorescent end can now emit.

The probes may be nucleic acids, as described herein, or nucleic acid derivatives. As used herein, a “nucleic acid derivative” is a non-naturally occurring nucleic acid or a unit thereof. Nucleic acid derivatives may contain non-naturally occurring elements such as non- naturally occurring nucleotides and non-naturally occurring backbone linkages. These include substituted purines and pyrimidines such as C-5 propyne modified bases, 5-methylcytosine, 2-aminopurine, 2-amino-6-chloropurine, 2,6-diaminopurine, hypoxanthine, 2-thiouracil and pseudoisocytosine. Other such modifications are well known to those of skill in the art.

The nucleic acid derivatives may also encompass substitutions or modifications, such as in the bases and/or sugars. For example, they include nucleic acids having backbone sugars which are covalently attached to low molecular weight organic groups other than a hydroxyl group at the 3′ position and other than a phosphate group at the 5′ position. Thus, modified nucleic acids may include a 2′-O-alkylated ribose group. In addition, modified nucleic acids may include sugars such as arabinose instead of ribose.

The probes if comprising nucleic acid components can be stabilized in part by the use of backbone modifications. The invention intends to embrace, in addition to the peptide and locked nucleic acids discussed herein, the use of the other backbone modifications such as but not limited to phosphorothioate linkages, phosphodiester modified nucleic acids, combinations of phosphodiester and phosphorothioate nucleic acid, methylphosphonate, alkylphosphonates, phosphate esters, alkylphosphonothioates, phosphoramidates, carbamates, carbonates, phosphate triesters, acetamidates, carboxymethyl esters, methylphosphorothioate, phosphorodithioate, p-ethoxy, and combinations thereof.

In some embodiments, the probe is a nucleic acid that is a peptide nucleic acid (PNA), a bisPNA clamp, a pseudocomplementary PNA, a locked nucleic acid (LNA), DNA, RNA, or co-nucleic acids of the above such as DNA-LNA co-nucleic acids. siRNA or miRNA or RNAi molecules can be similarly used.

In some embodiments, the probe is a peptide nucleic acid (PNA), a bisPNA clamp, a locked nucleic acid (LNA), a ssPNA, a pseudocomplementary PNA (pcPNA), a two-armed PNA (as described in co-pending U.S. patent application having Ser. No. 10/421,644 and publication number US 2003-0215864 A1 and published Nov. 20, 2003, and PCT application having serial number PCT/US03/12480 and publication number WO 03/091455 A1 and published Nov. 6, 2003, filed on Apr. 23, 2003), or co-polymers thereof (e.g., a DNA-LNA co-polymer).

Other backbone modifications, particularly those relating to PNAs, include peptide and amino acid variations and modifications. Thus, the backbone constituents of PNAs may be peptide linkages, or alternatively, they may be non-peptide linkages. Examples include acetyl caps, amino spacers such as 8-amino-3,6-dioxaoctanoic acid (referred to herein as 0-linkers), amino acids such as lysine (particularly useful if positive charges are desired in the PNA), and the like. Various PNA modifications are known and probes incorporating such modifications are commercially available from sources such as Boston Probes, Inc.

As stated herein, the agent (e.g., the polymer) may be labeled. As an example, if the agent is a nucleic acid, it may be labeled through the use of sequence-specific probes that bind to the polymer in a sequence-specific manner. The sequence-specific probes are labeled with a detectable label (e.g., a fluorophore or a radioisotope). The nucleic acid however can also be synthesized in a manner that incorporates fluorophores directly into the growing nucleic acid. For example, this latter labeling can be accomplished by chemical means or by the introduction of active amino or thiol groups into nucleic acids. (Proudnikov and Mirabekov, Nucleic Acid Research, 24:4535-4532, 1996.) An extensive description of modification procedures that can be performed on a nucleic acid polymer can be found in Hermanson, G. T., Bioconjugate Techniques, Academic Press, Inc., San Diego, 1996, which is incorporated by reference herein.

There are several known methods of direct chemical labeling of DNA (Hermanson, 1996; Roget et al., 1989; Proudnikov and Mirabekov, 1996). One of the methods is based on the introduction of aldehyde groups by partial depurination of DNA. Fluorescent labels with an attached hydrazine group are efficiently coupled with the aldehyde groups and the hydrazine bonds are stabilized by reduction with sodium labeling efficiencies around 60%. The reaction of cytosine with bisulfite in the presence of an excess of an amine fluorophore leads to transamination at the N4 position (Hermanson, 1996). Reaction conditions such as pH, amine fluorophore concentration, and incubation time and temperature affect the yield of products formed. At high concentrations of the amine fluorophore (3M), transamination can approach 100% (Draper and Gold, 1980).

In addition to the above method, it is also possible to synthesize nucleic acids de novo (e.g., using automated nucleic acid synthesizers) using fluorescently labeled nucleotides. Such nucleotides are commercially available from suppliers such as Amersham Pharmacia Biotech, Molecular Probes, and New England Nuclear/Perkin Elmer.

Probes are generally labeled with a detectable label. A detectable label is a moiety, the presence of which can be ascertained directly or indirectly. Generally, detection of the label involves the creation of a detectable signal such as for example an emission of energy. The label may be of a chemical, peptide or nucleic acid nature although it is not so limited. The nature of label used will depend on a variety of factors, including the nature of the analysis being conducted, the type of the energy source and detector used and the type of polymer and probe. The label should be sterically and chemically compatible with the constituents to which it is bound.

The label can be detected directly for example by its ability to emit and/or absorb electromagnetic radiation of a particular wavelength. A label can be detected indirectly for example by its ability to bind, recruit and, in some cases, cleave another moiety which itself may emit or absorb light of a particular wavelength (e.g., an epitope tag such as the FLAG epitope, an enzyme tag such as horseradish peroxidase, etc.). Generally the detectable label can be selected from the group consisting of directly detectable labels such as a fluorescent molecule (e.g., fluorescein, rhodamine, tetramethylrhodamine, R-phycoerythrin, Cy-3, Cy-5, Cy-7, Texas Red, Phar-Red, allophycocyanin (APC), fluorescein amine, eosin, dansyl, umbelliferone, 5-carboxyfluorescein (FAM), 2′7′-dimethoxy-4′5′-dichloro-6-carboxyfluorescein (JOE), 6 carboxyrhodamine (R6G), N,N,N′,N′-tetramethyl-6-carboxyrhodamine (TAMRA), 6-carboxy-X-rhodamine (ROX), 4-(4′-dimethylaminophenylazo) benzoic acid (DABCYL), 5-(2′-aminoethyl) aminonaphthalene-1-sulfonic acid (EDANS), 4-acetamido-4′-isothiocyanatostilbene-2, 2′ disulfonic acid, acridine, acridine isothiocyanate, r-amino-N-(3-vinylsulfonyl)phenylnaphthalimide-3,5, disulfonate (Lucifer Yellow VS), N-(4-anilino-1-naphthyl)maleimide, anthranilamide, Brilliant Yellow, coumarin, 7-amino-4-methylcoumarin, 7-amino-4-trifluoromethylcouluarin (Coumarin 151), cyanosine, 4′, 6-diaminidino-2-phenylindole (DAPI), 5′, 5″-diaminidino-2-phenylindole (DAPI), 5′, 5″-dibromopyrogallol-sulfonephthalein (Bromopyrogallol Red), 7-diethylamino-3-(4′-isothiocyanatophenyl)-4-methylcoumarin diethylenetriamine pentaacetate, 4,4′-diisothiocyanatodihydro-stilbene-2, 2′-disulfonic acid, 4,4′-diisothiocyanatostilbene-2, 2′-disulfonic acid, 4-dimethylaminophenylazophenyl-4′-isothiocyanate (DABITC), eosin isothiocyanate, erythrosin B, erythrosin isothiocyanate, ethidium, 5-(4,6-dichlorotriazin-2-yl) aminofluorescein (DTAF), QFITC (XRITC), fluorescamine, IR144, IR1446, Malachite Green isothiocyanate, 4-methylumbelliferone, ortho cresolphthalein, nitrotyrosine, pararosaniline, Phenol Red, B-phycoerythrin, o-phthaldialdehyde, pyrene, pyrene butyrate, succinimidyl 1-pyrene butyrate, Reactive Red 4 (Cibacron. RTM. Brilliant Red 3B-A), lissamine rhodamine B sulfonyl chloride, rhodamine B, rhodamine 123, rhodamine X, sulforhodamine B, sulforhodamine 101, sulfonyl chloride derivative of sulforhodamine 101, tetramethyl rhodamine, riboflavin, rosolic acid, and terbium chelate derivatives), a chemiluminescent molecule, a bioluminescent molecule, a chromogenic molecule, a radioisotope (e.g., p³² or H³, ¹⁴C, ¹²⁵I and ¹³¹I), an electron spin resonance molecule (such as for example nitroxyl radicals), an optical or electron density molecule, an electrical charge transducing or transferring molecule, an electromagnetic molecule such as a magnetic or paramagnetic bead or particle, a semiconductor nanocrystal or nanoparticle (such as quantum dots described for example in U.S. Pat. No. 6,207,392 and commercially available from Quantum Dot Corporation and Evident Technologies), a colloidal metal, a colloid gold nanocrystal, a nuclear magnetic resonance molecule, and the like.

The detectable label can also be selected from the group consisting of indirectly detectable labels such as an enzyme (e.g., alkaline phosphatase, horseradish peroxidase, β-galactosidase, glucoamylase, lysozyme, luciferases such as firefly luciferase and bacterial luciferase (U.S. Pat. No. 4,737,456); saccharide oxidases such as glucose oxidase, galactose oxidase, and glucose-6-phosphate dehydrogenase; heterocyclic oxidases such as uricase and xanthine oxidase coupled to an enzyme that uses hydrogen peroxide to oxidize a dye precursor such as HRP, lactoperoxidase, or microperoxidase), an enzyme substrate, an affinity molecule, a ligand, a receptor, a biotin molecule, an avidin molecule, a streptavidin molecule, an antigen (e.g., epitope tags such as the FLAG or HA epitope), a hapten (e.g., biotin, pyridoxal, digoxigenin fluorescein and dinitrophenol), an antibody, an antibody fragment, a microbead, and the like. Antibody fragments include Fab, F(ab)₂, Fd and antibody fragments which include a CDR3 region.

In some embodiments, the detectable label is a member of a FRET fluorophore pair. FRET fluorophore pairs are two fluorophores that are capable of undergoing FRET to produce or eliminate a detectable signal when positioned in proximity to one another. Examples of donors include Alexa 488, Alexa 546, BODIPY 493, Oyster 556, Fluor (FAM), Cy3 and TMR (Tamra). Examples of acceptors include Cy5, Alexa 594, Alexa 647 and Oyster 656. Cy5 can work as a donor with Cy3, TMR or Alexa 546, as an example. FRET should be possible with any fluorophore pair having fluorescence maxima spaced at 50-100 nm from each other.

The polymer may be labeled in a sequence non-specific manner. For example, if the polymer is a nucleic acid such as DNA, then its backbone may be stained with a backbone label. Examples of backbone stains that label nucleic acids in a sequence non-specific manner include intercalating dyes such as phenanthridines and acridines (e.g., ethidium bromide, propidium iodide, hexidium iodide, dihydroethidium, ethidium homodimer-1 and -2, ethidium monoazide, and ACMA); minor grove binders such as indoles and imidazoles (e.g., Hoechst 33258, Hoechst 33342, Hoechst 34580 and DAPI); and miscellaneous nucleic acid stains such as acridine orange (also capable of intercalating), 7-AAD, actinomycin D, LDS751, and hydroxystilbamidine. All of the aforementioned nucleic acid stains are commercially available from suppliers such as Molecular Probes, Inc.

Still other examples of nucleic acid stains include the following dyes from Molecular Probes: cyanine dyes such as SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red).

As used herein, “conjugated” means two entities stably bound to one another by any physiochemical means. It is important that the nature of the attachment is such that it does not substantially impair the effectiveness of either entity. Keeping these parameters in mind, any covalent or non-covalent linkage known to those of ordinary skill in the art may be employed. In some embodiments, covalent linkage is preferred. Noncovalent conjugation includes hydrophobic interactions, ionic interactions, high affinity interactions such as biotin-avidin and biotin-streptavidin complexation and other affinity interactions. Such means and methods of attachment are known to those of ordinary skill in the art.

The various components described herein can be conjugated to each other by any mechanism known in the art. For instance, functional groups which are reactive with various labels include, but are not limited to (functional group- reactive group of light emissive compound), activated ester:amines or anilines; acyl azide:amines or anilines; acyl halide: amines, anilines, alcohols or phenols; acyl nitrile:alcohols or phenols; aldehyde:amines or anilines; alkyl halide:amines, anilines, alcohols, phenols or thiols; alkyl sulfonate:thiols, alcohols or phenols; anhydride:alcohols, phenols, amines or anilines; aryl halide:thiols; aziridine:thiols or thioethers; carboxylic acid:amines, anilines, alcohols or alkyl halides; diazoalkane:carboxylic acids; epoxide:thiols; haloacetamide:thiols; halotriazine:amines, anilines or phenols; hydrazine:aldehydes or ketones; hydroxyamine:aldehydes or ketones; imido ester:amines or anilines; isocyanate:amines or anilines; and isothiocyanate:amines or anilines.

Linkers can be any of a variety of molecules, preferably nonactive, such as nucleotides or multiple nucleotides, straight or even branched saturated or unsaturated carbon chains of C₁-C₃₀, phospholipids, amino acids, and in particular glycine, and the like, whether naturally occurring or synthetic. Additional linkers include alkyl and alkenyl carbonates, carbamates, and carbamides. These are all related and may add polar functionality to the linkers such as the C₁-C₃₀ previously mentioned. As used herein, the terms linker and spacer are used interchangeably.

A wide variety of spacers can be used, many of which are commercially available, for example, from sources such as Boston Probes, Inc. (now Applied Biosystems). Spacers are not limited to organic spacers, and rather can be inorganic also (e.g., —O—Si—O—, or O—P—O—). Additionally, they can be heterogeneous in nature (e.g., composed of organic and inorganic elements). Essentially, any molecule having the appropriate size restrictions and capable of being linked to the various components such as fluorophore and probe can be used as a linker. Examples include the E linker (which also functions as a solubility enhancer), the X linker which is similar to the E linker, the 0 linker which is a glycol linker, and the P linker which includes a primary aromatic amino group (all supplied by Boston Probes, Inc., now Applied Biosystems). Other suitable linkers are acetyl linkers, 4-aminobenzoic acid containing linkers, Fmoc linkers, 4-aminobenzoic acid linkers, 8-amino-3, 6-dioxactanoic acid linkers, succinimidyl maleimidyl methyl cyclohexane carboxylate linkers, succinyl linkers, and the like. Another example of a suitable linker is that described by Haralambidis et al. in U.S. Pat. No. 5,525,465, issued on Jun. 11, 1996.

The conjugations or modifications described herein employ routine chemistry, which is known to those skilled in the art of chemistry. The use of linkers such as mono- and hetero-bifunctional linkers is documented in the literature (e.g., Hermanson, 1996) and will not be repeated here.

The linker molecules may be homo-bifunctional or hetero-bifunctional cross-linkers, depending upon the nature of the molecules to be conjugated. Homo-bifunctional cross-linkers have two identical reactive groups. Hetero-bifunctional cross-linkers are defined as having two different reactive groups that allow for sequential conjugation reaction. Various types of commercially available cross-linkers are reactive with one or more of the following groups: primary amines, secondary amines, sulphydryls, carboxyls, carbonyls and carbohydrates. Examples of amine-specific cross-linkers are bis(sulfosuccinimidyl) suberate, bis[2-(succinimidooxycarbonyloxy)ethyl] sulfone, disuccinimidyl suberate, disuccinimidyl tartarate, dimethyl adipimate-2 HCl, dimethyl pimelimidate-2 HCl, dimethyl suberimidate-2 HCl, and ethylene glycolbis-[succinimidyl- [succinate]]. Cross-linkers reactive with sulfhydryl groups include bismaleimidohexane, 1,4-di-[3′-(2′-pyridyldithio)-propionamido)] butane, 1-[p-azidosalicylamido]-4- [iodoacetamido] butane, and N-[4-(p-azidosalicylamido) butyl]-3′-[2′-pyridyldithio] propionamide. Cross-linkers preferentially reactive with carbohydrates include azidobenzoyl hydrazine. Cross-linkers preferentially reactive with carboxyl groups include 4-[p-azidosalicylamido] butylamine. Heterobifunctional cross-linkers that react with amines and sulfhydryls include N-succinimidyl-3-[2-pyridyldithio] propionate, succinimidyl [4-iodoacetyl]aminobenzoate, succinimidyl 4-[N-maleimidomethyl] cyclohexane-1- carboxylate, m-maleimidobenzoyl-N-hydroxysuccinimide ester, sulfosuccinimidyl 6-[3-[2-pyridyldithio]propionamido]hexanoate, and sulfosuccinimidyl 4-[N-maleimidomethyl] cyclohexane-1-carboxylate. Heterobifinctional cross-linkers that react with carboxyl and amine groups include 1-ethyl-3-[3-dimethylaminopropyl]-carbodiimide hydrochloride. Heterobifunctional cross-linkers that react with carbohydrates and sulfhydryls include 4-[N-maleimidomethyl]-cyclohexane-1-carboxylhydrazide-2 HCl, 4-(4-N-maleimidophenyl)-butyric acid hydrazide-2HCl, and 3-[2-pyridyldithio] propionyl hydrazide. The cross-linkers are bis-[β-4-azidosalicylamido)ethyl]disulfide and glutaraldehyde.

Amine or thiol groups may be added at any nucleotide of a synthetic nucleic acid so as to provide a point of attachment for a bifunctional cross-linker molecule. The nucleic acid may be synthesized incorporating conjugation-competent reagents such as Uni-Link AminoModifier, 3′-DMT-C6-Amine-ON CPG, AminoModifier II, N-TFA-C6-AminoModifier, C6-ThiolModifier, C6-Disulfide Phosphoramidite and C6-Disulfide CPG (Clontech, Palo Alto, Calif.).

Non-covalent methods of conjugation may also be used to bind a detectable label to a probe, for example. Non-covalent conjugation includes hydrophobic interactions, ionic interactions, high affinity interactions such as biotin-avidin and biotin-streptavidin complexation and other affinity interactions. As an example, a molecule such as avidin may be attached the nucleic acid, and its binding partner biotin may be attached to the probe.

In some instances, it may be desirable to use a linker or spacer comprising a bond that is cleavable under certain conditions. For example, the bond can be one that cleaves under normal physiological conditions or that can be caused to cleave specifically upon application of a stimulus such as light. Readily cleavable bonds include readily hydrolyzable bonds, for example, ester bonds, amide bonds and Schiff's base-type bonds. Bonds which are cleavable by light are known in the art.

The agent (e.g., the polymer) may be analyzed using a single molecule analysis system (e.g., a single polymer analysis system). A single molecule detection system is capable of analyzing single molecules separately from other molecules. Such a system may be capable of analyzing single molecules in a linear manner and/or in their totality. In certain embodiments in which detection is based predominately on the presence or absence of a signal, linear analysis may not be required. However, there are other embodiments embraced by the invention which would benefit from the ability to analyze linearly molecules (preferably nucleic acids) in a sample. These include applications in which the sequence of the nucleic acid is desired, or in which the polymers are distinguished based on spatial labeling pattern rather than a unique detectable label.

Thus, the polymers can be analyzed using linear polymer analysis systems. A linear polymer analysis system is a system that analyzes polymers such as nucleic acids, in a linear manner (i.e., starting at one location on the polymer and then proceeding linearly in either direction therefrom). As a polymer is analyzed, the detectable labels attached to it are detected in either a sequential or simultaneous manner. When detected simultaneously, the signals usually form an image of the polymer, from which distances between labels can be determined. When detected sequentially, the signals are viewed in histogram (signal intensity vs. time) that can then be translated into a map, with knowledge of the velocity of the polymer. It is to be understood that in some embodiments, the polymer is attached to a solid support, while in others it is free flowing. In either case, the velocity of the polymer as it moves past, for example, an interaction station or a detector, will aid in determining the position of the labels relative to each other and relative to other detectable markers that may be present on the polymer.

An example of a suitable system is the GeneEngine™ (U.S. Genomics, Inc., Woburn, Mass.). The Gene Engine™ system is described in PCT patent applications W098/35012 and WO00/09757, published on Aug. 13, 1998, and Feb. 24, 2000, respectively, and in issued U.S. Pat. No. 6,355,420 B1, issued Mar. 12, 2002. The contents of these applications and patent, as well as those of other applications and patents, and references cited herein are incorporated by reference herein in their entirety. This system is both a single molecule analysis system and a linear polymer analysis system. It allows, for example, single nucleic acids to be passed through an interaction station in a linear manner, whereby the nucleotides in the nucleic acid are interrogated individually in order to determine whether there is a detectable label conjugated to the nucleic acid. Interrogation involves exposing the nucleic acid to an energy source such as optical radiation of a set wavelength. The mechanism for signal emission and detection will depend on the type of label sought to be detected, as described herein.

The systems described herein will encompass at least one detection system. The nature of such detection systems will depend upon the nature of the detectable label. The detection system can be selected from any number of detection systems known in the art. These include an electron spin resonance (ESR) detection system, a charge coupled device (CCD) detection system, a fluorescent detection system, an electrical detection system, a photographic film detection system, a chemiluminescent detection system, an enzyme detection system, an atomic force microscopy (AFM) detection system, a scanning tunneling microscopy (STM) detection system, an optical detection system, a nuclear magnetic resonance (NMR) detection system, a near field detection system, and a total internal reflection (TIR) detection system, many of which are electromagnetic detection systems.

Other single molecule nucleic acid analytical methods can also be used to analyze nucleic acid targets following the chamber based processing of the invention. These include fiber-fluorescence in situ hybridization (fiber-FISH) (Bensimon, A. et al., Science 265(5181):2096-2098 (1997)). In fiber-FISH, nucleic acid molecules are elongated and fixed on a surface by molecular combing. Hybridization with fluorescently labeled probe sequences allows determination of sequence landmarks on the nucleic acid molecules. The method requires fixation of elongated molecules so that molecular lengths and/or distances between markers can be measured. Pulse field gel electrophoresis can also be used to analyze the labeled nucleic acid molecules. Pulse field gel electrophoresis is described by Schwartz, D. C. et al., Cell 37(1):67-75 (1984). Other nucleic acid analysis systems are described by Otobe, K. et al., Nucleic Acids Res. 29(22):E109 (2001), Bensimon, A. et al. in U.S. Pat. No. 6,248,537, issued Jun. 19, 2001, Herrick, J. et al., Chromosome Res. 7(6):409:423 (1999), Schwartz in U.S. Pat. No. 6,150,089 issued Nov. 21, 2000 and U.S. Pat. No. 6,294,136, issued Sep. 25, 2001. Other linear polymer analysis systems can also be used, and the invention is not intended to be limited to solely those listed herein.

### EXAMPLES

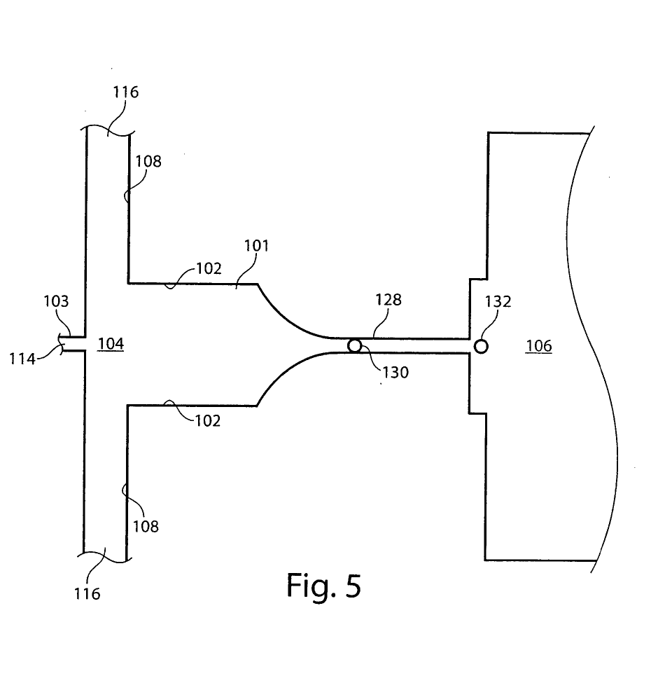

FIG. 5 shows a microchannel 101 configuration used during a first experiment that relates to diffusion mediated cleanup of a carrier fluid. The microchannel has an upstream portion 104 that is 100 microns wide and that is in fluid communication with an approximately 2 micron wide carrier fluid introduction channel 103. A pair of 35 micron wide sheathing fluid introduction channels 108 are also in fluid communication with the upstream portion of the microchannel. The microchannel 101 narrows, in a funnel like configuration, to a width of 2 microns at point that is 200 microns downstream from the carrier fluid introduction channel. The 2 micron wide microchannel extends for about 160 microns until the channel abruptly widens to a 100 micron width. This 100 micron wide portion 128 of the channel extends downstream about 20 microns, where the channel abruptly widens again to a width of about 500 microns.

The microchannel shown in FIG. 5 is embedded on a microchip (not shown). Carrier fluid 114 and sheathing fluids 116 were provided to the chip at 14 psi. A 6 psi pressure was applied to the channel, at a position downstream from the 500 micron wide portion of the microchannel. These pressures resulted in fluid flow velocity of 20 microns per millisecond through the 2 micron portion 128 of the microchannel.

During a first portion of the experiment, carrier fluid containing DNA with probes bound thereto was passed into the microchannel 101 through the carrier fluid introduction channel 103. Sheathing fluids 116 were also introduced to the microchannel through the sheathing fluid introduction channels 108. Using a wide field imaging device, an image was taken of the DNA passing through the 2 micron wide portion of the microchannel at a first detection zone 130, that was 200 microns downstream from the carrier fluid introduction channel 103. The DNA distribution that was identified had a Gaussian profile with a full width half maximum (FWHM) of 0.46 microns, although 0.46 microns was the resolution limitation of the wide field imaging device.

During a second portion of the experiment, a wide field imaging device was positioned at a second detection zone 132 located downstream from the 2 micron wide portion of the microchannel, where the channel abruptly widens to a 100 micron wide channel. Again, a carrier fluid 114 containing DNA bound with probes and a pair of sheathing fluids 116 were passed down the microchannel 101. Images at the second detection zone 132 revealed DNA spanning across the channel in a Gaussian profile having a FWHM of 5 microns, or equivalently the central 5% of the 100 micron wide microchannel. The functional channel width at the second detection zone is estimated to be about 80 microns, when boundary layer conditions with the walls of the channel are considered. This suggest that the DNA resided within 6.3% of the functional width of the microchannel. This percentage suggests all of the DNA may have equivalently passed through a detection zone having a 0.13 micron diameter, such as might be associated with a single point detection zone centered at the position of the at the first detection zone 130. This is equivalently 6.3% of the 2 micron wide portion of the microchannel.

During a third portion of the experiment, a carrier fluid 114 containing a single organic dye (Cy3), which diffuses at a rate similar to many probes, was delivered to the microchannel from the carrier fluid introduction channel 103. Sheathing fluids were also introduced to the microchannel. A wide field imaging device was used to detect the dye passing through the first detection zone in the 2 micron wide portion of the microchannel. The images revealed that the dye had diffused to a homogenous distribution across the entire 2 micron wide portion 128 of the microchannel.

The final step of the experiment involved calculating the cleanup factor that would be achieved by the microchannel of FIG. 5. An estimate of the laser excitation signal (i.e., a Gaussian beam with a FWHM of 0.3 microns defined by exp(−2.773*(x/0.3)2)) is convoluted with each of the DNA and probe distributions that were identified with the wide field imaging device. The convolution results are representative of the signals that would be received by a point detector centered at the first detection zone. The laser excitation signal when convolved with a Gaussian provile having a full width half maximum of 0.13 microns, like that associated with DNA passing through a 2 micron detection zone, results in a value of 0.92. The excitation signal convolved with a 2 micron square wave, like that associated with probes passing through a detection zone that spans the full width of the 2 micron channel, results in a value of 0.16. In this sense, the experiment shows that diffusion mediated cleanup in the microchannel of FIG. 5, with a 0.3 micron excitation laser beam centered in the first detection zone results in a cleanup factor of about 6x, when the resulting value (0.92) of DNA with probes bound thereto is compared with that of free probes (0.16).

### Equivalents

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the invention. The advantages and objects of the invention are not necessarily encompassed by each embodiment of the invention.

All references, patents and patent applications that are recited in this application are incorporated by reference herein in their entirety. In case of conflict, the present specification, including definitions, will control.

What is claimed is:

1. A microfluidic apparatus comprising:
a microchannel having an upstream portion and a downstream portion, the microchannel constructed and arranged to transport a carrier fluid such that, when present in the carrier fluid, targets and non-targets flow from the upstream portion toward the downstream portion;
a first sheathing fluid introduction channel adapted to provide a first sheathing fluid to the microchannel such that non-targets can diffuse from the carrier fluid to the first sheathing fluid; and
a sample capture channel located downstream from the first sheathing fluid introduction channel and adapted to receive the carrier fluid after at least a portion of the non-targets have diffused from the carrier fluid and into the first sheathing fluid.

2.-36. (canceled)

37. A method of removing non-targets from a carrier fluid that contains targets, the method comprising:
providing a microchannel adapted to deliver the carrier fluid from an upstream portion toward a downstream portion of the microchannel;
providing the carrier fluid to the upstream portion of the microchannel, the carrier fluid containing a plurality of targets and a plurality of non-targets;
providing a first sheathing fluid to the microchannel such that the first sheathing fluid flows from the upstream portion toward the downstream portion adjacent to the carrier fluid;
diffusing a first portion of the plurality of non-targets from the carrier fluid to the first sheathing fluid; and
passing the carrier fluid through a sample capture channel after the first portion of the targets have diffused from the carrier fluid and into the first sheathing fluid.

38. The method of claim 37, wherein the sample capture channel is constructed and arranged and conditions are such that the first portion is at least 60% (0.60) of the plurality of non-targets provided to the microchannel.

39. The method of claim 37, wherein the sample capture channel is constructed and arranged and conditions are such that the first portion is at least 85% (0.85) of the plurality of non-targets provided to the microchannel.

40. The method of claim 37, further comprising:
retaining within the carrier fluid at least 80% (0.80) of the plurality of targets provided to the microchannel as the carrier fluid is passed through the sample capture channel.

41. The method of claim 37, further comprising:
retaining within the carrier fluid at least 90% (0.90) of the plurality of targets provided to the microchannel as the carrier fluid is passed through the sample capture channel.

42. The method of claim 37, wherein providing the first sheathing fluid comprises providing a pair of opposed flows of sheathing fluid to the microchannel.

43. The method of claim 42, further comprising:
creating a velocity gradient within the carrier fluid with the pair of opposed flows of sheathing fluid.

44. The method of claim 37, further comprising:
removing at least a portion of the first sheathing fluid from the microchannel such that the first portion of non-targets contained therein is removed from the microchannel.

45. The method of claim 37, wherein removing at least a portion of the first sheathing fluid comprises removing substantially all of the first sheathing fluid.

46. The method of claim 37, wherein removing at least a portion of the first sheathing fluid comprises removing a portion of the carrier fluid.

47. The method of claim 37, further comprising:
providing a second sheathing fluid to the microchannel;
diffusing a second portion of the plurality of non-targets diffuse from the carrier fluid to the second sheathing fluid; and
passing the carrier fluid through a second sample capture channel after the second portion of the targets have diffused from the carrier fluid and into the second sheathing fluid.

48. The method of claim 47, wherein the second sample capture channel is constructed and arranged such that the carrier fluid passed through the second sample capture channel contains fewer than 90% (0.90) of the non-targets provided to the microchannel after the second portion of non-targets has diffused to the second sheathing fluid.

49. The method of claim 47, wherein the second sample capture channel is constructed and arranged such that the carrier fluid passed through the second sample capture channel contains fewer than 95% (0.95) of the non-targets provided to the microchannel after the second portion of non-targets has diffused to the second sheathing fluid.

50. The method of claim 47, wherein the second sample capture channel is constructed and arranged such that the carrier fluid passed through the second sample capture channel contains more than 80% (0.80) of the targets provided to the microchannel.

51. The method of claim 47, wherein the second sample capture channel is constructed and arranged such that the carrier fluid passed through the second sample capture channel contains more than 90% of the targets provided to the microchannel.

52. The method of claim 47, further comprising:
removing at least a portion of the second sheathing fluid from the microchannel such that non-targets contained therein are removed from the microchannel; and then providing a third sheathing fluid to the microchannel such that at least a third portion of the plurality of non-targets diffuse from the carrier fluid to the third sheathing fluid.

53. The method of claim 37, wherein the carrier fluid moves in the microchannel with a velocity between 0.1 and 20.0 mm/second.

54. The method of claim 37, wherein the plurality of targets include polymers.

55.-56. (canceled)

57. The method of claim 37, wherein the plurality of targets include nucleic acids.

58.-67. (canceled)
